(19) United States
(12) Patent Application Publication
(10) Pub. No.: US 2011/0207166 A1
(43) Pub. Date: Aug. 25, 2011

(54) Human bone marrow microenvironments and uses thereof

(76) Inventor: Sarah Rivkah Vaiselbuh, Brooklyn, NY (US)

(21) Appl. No.: 12/927,067

(22) Filed: Nov. 5, 2010

Related U.S. Application Data
(60) Provisional application No. 61/280,639, filed on Nov. 6, 2009.

(51) Int. Cl. C12Q 1/18; C12N 5/0775; C12N 5/09; A01K 67/00; A61K 35/12

(52) U.S. Cl. 435/32; 435/366; 800/8; 424/93.7

(57) ABSTRACT

The present invention is directed to an in vitro cultured permissive niche, or human bone marrow microenvironment, comprising a scaffold coated with human mesenchymal stem cells and a culture medium, wherein the stem cells are viable and proliferate in culture and the niche is permissive for the establishment of introduced hematopoietic or leukemic cell populations. The present invention is also directed to establishment of a permissive niche in a non-human animal model comprising a scaffold coated with human mesenchymal stem cells introduced into the animal ectopically, wherein the niche and the model are permissive for the establishment of introduced hematopoietic or leukemic cell populations. The implanted scaffold forms an ectopic human bone marrow microenvironment to study the mesenchymal leukemic stem cell niche. In addition, the present invention is directed to methods of using the in vitro cultured human bone marrow microenvironment and the non-human animal model to evaluate an agent for anti-leukemic properties.

### CROSS-REFERENCE TO RELATED APPLICATION

This application claims the benefit of U.S. Provisional Patent Application No. 61/280,639, filed on Nov. 6, 2009, the content of which is herein incorporated by reference.

### FIELD OF THE INVENTION

The present invention relates to the human bone marrow microenvironments both in vitro and in vivo, and their uses for the evaluation of the anti-leukemic properties of agents.

### BACKGROUND OF THE INVENTION

Stem cells—cells that have the potential to regenerate tissue over a life time—are defined by their cell biological characteristics such as proliferation and differentiation, quiescence, self-renewal and anti-apoptosis. However, the mechanisms that guide stem cells into the decision to remain quiescent or exit the cell-cycle for self renewal and differentiation remain unclear.

Stem cells appear to be a functionally heterogeneous population that lives in cellular neighborhoods, called the stem cell niche [1]. The stem cell niche is defined as the habitat of stem cells within the bone marrow (BM) ecosystem, securing their longevity and ‘stemness’. Schofield postulated that the stem cell becomes essentially a ‘fixed tissue’ cell in association with other neighboring cells and extracellular matrix which determine its behavior in an anatomical three-dimensional place called a niche [2]. The stem cell niche provides a micro-cosmos that is both permissive and instructive for stem cell signaling and as such offers a unique target for the development of novel stem cell therapeutics.

Different components of the specific BM microenvironment that guide hematopoietic stem cells (HSC) have been identified [3, 4, 5], but the niche for malignant hematopoiesis remains to be elucidated. Acute myeloid leukemia (AML) is a clinically heterogeneous disease with variable treatment outcome and about 25% relapse rate. One of the proposed mechanisms of chemoresistance in leukemia involves the interaction with stromal cell components of the niche, mediated by very late antigen (VLA-4) on leukemic cells to fibronectin on BM stromal cells [6]. In addition, the chemokine stromal-cell derived factor (SDF-1/CXCL12) and its receptor CXCR4 are critical for engraftment of normal human repopulating stem cells in SCID mice [7] as well as for homing and migration of AML blasts. SDF-1/CXCL12, produced by BM stromal cells, regulates stem cell niche maintenance, stem cell trafficking and the cell cycle via its receptor CXCR4 [8, 9] and CXCR4 expression promotes leukemia cell survival and adhesion [10]. The prime site for minimal residual disease (MRD) in leukemia is presumed to be the BM milieu. However, due to the plasticity of the stromal compartment and the lack of stromal cell specific markers, our knowledge of stromal niche biology is still very limited.

In 1924, Russia-born morphologist Alexander A. Maximov used histological observations to identify a singular type of precursor cell within the mesenchyme which develops into different types of blood, in support of his “Unitarian” theory of hematopoiesis [11]. Scientists Ernest A. McCulloch and James E. Till first revealed the clonal nature of stromal marrow cells in the 60's [12]. An ex-vivo assay for examining the clonal potential of multipotent marrow stromal cells was reported by Friedenstein and his team in the 1970's. They developed an assay system wherein stromal cells were referred to as colony-forming unit fibroblasts (CFU-f) [13]. It was Dexter and colleagues in 1977 who first described, in long-term BM cultures, a type of stromal cell called the blanket cell that was capable of cobble-stone area formation [14, 15]. Cobble-stone areas are formed when hematopoietic progenitors migrate underneath the blanket cell and become phase-dim, creating the typical ‘cobble-stone’ appearance. As such, cobble-stone forming units represent areas of active hematopoiesis within the two-dimensional stroma in long-term BM cultures.

During embryonic development the mesoangioblasts, originating at the dorsal aorta of the embryo, are considered vessel-associated stem cells which can give rise to differentiated mesodermal cell types including smooth muscle cells, bone, cartilage and adipocytes [16]. Mesoangioblasts may represent ancestors of undifferentiated mesenchymal stem cells (MSC) in postnatal life. Adult MSC have retained much of the differential potential displayed during embryonic life, likely due to their mesodermal origin. Adipocytes, chondrocytes and myocytes have been derived from adult BM-derived MSC in tissue culture and throughout the body, MSC form the supportive structure in which the functional cells of a specific tissue reside. Because of their multipotentiality and their physical location in the perivascular space, MSC may prove useful for repair and regeneration of marrow stroma by the production of growth factors and cytokines with autocrine and paracrine activities [17].

There exists a present need for new methods and assays for identifying agents having anti-leukemic properties, targeting the leukemic cells as well as the bone marrow niche and for assessing the ability of agents to be therapeutically effective in the treatment of leukemia for specific patients. The present invention satisfies this need.

### SUMMARY OF THE INVENTION

The present invention is directed to an in vitro cultured human bone marrow microenvironment comprising a scaffold coated with human mesenchymal stem cells and a culture medium, wherein the stem cells are viable and proliferate in culture.

The present invention is also directed to a non-human animal comprising a scaffold coated with human mesenchymal stem cells introduced into the animal ectopically.

In addition, the present invention is directed to a method of making an in vitro human bone marrow microenvironment comprising culturing a scaffold with human mesenchymal stem cells under conditions permitting the stem cells to coat the scaffold.

The present invention also provides a method of making a non-human animal model comprising the steps of: a) culturing a scaffold with human mesenchymal stem cells under conditions permitting the stem cells to coat the scaffold; and b) introducing the scaffold coated with the human mesenchymal stem cells into the non-human animal ectopically.

Also provided by the present invention is a method for evaluating an agent for anti-leukemic properties comprising the steps of: a) obtaining or preparing in vitro human bone marrow microenvironment in which leukemia cells are established; b) contacting the agent with the microenvironment; and c) evaluating the anti-leukemic properties of the agent.

In addition, the present invention provides a method for evaluating an agent for anti-leukemic properties comprising the steps of: a) obtaining or preparing the non-human animal; b) introducing the agent into the non-human animal; and c) evaluating the anti-leukemic properties of the agent.

Still further, the present invention provides a use of the in vitro cultured human bone marrow microenvironment or the non-human animal model, as a model to study human bone marrow development, to study leukemia in the mesenchymal stem cell niche, to study leukemia cell biology, and to study the anti-leukemic properties of an agent.

Additional objects of the invention will be apparent from the description which follows.

### BRIEF DESCRIPTION OF THE FIGURES

FIG. 1: Phenotypic characterization of MSC. (A) Flowcytometry: MSC niche cells are CD90, CD105 and CD146 positive and CD34/CD45 negative. MSC subset expresses also CXCR4 (1.26%). (B) Immunohistochemistry: MSC stain positive for CD90, CD105 and CD146. MSC forms cobblestone areas and provide niches for hematopoietic progenitors (arrows). (Phase contract microscopy—DAB Peroxidase stain/brown).

FIG. 2: Phenotypic characterization of Acute Myeloid Leukemia (AML). (A) Flowcytometry: Primary AML cells (leukapheresis product) are sorted for CD45 (99.5%). An immature CD45+/CD34−/CD38− AML population was selected (1.3%) and 65.5% of these cells express CXCR4. (B) Cell Cycle Analysis: Cell cycle analysis of these AML subpopulations reveals that the AML/CXCR negative population is in the G1/G2 phase of the cell cycle, while the AML/CXCR4 positive subset is in the G0/quiescent phase.

FIG. 3: Ultrastructural evaluation of the AML-MSC interaction. (A) Light microscopy of empty polyurethane scaffold. (B) Two-dimensional tissue culture: Phase contrast microscopy of primary AML cells that attach (1), migrate underneath (2) and form pseudo-uropods underneath the MSC (3) (32×). (C) Three-dimensional tissue culture: Electron microscopy: MSC adheres to the scaffold (s) and AML cell forms pseudo-uropod (P) to attach to the MSC on the scaffold. (4000×). Intimate surface contact is noticed between the pseudo-uropod from the AML cell and the MSC cell. Granulocytic cytoplasm of the AML cell migrates underneath the MSC (6700×). Higher magnification of the AML-MSC interaction (arrows) (27000×).

FIG. 4: In vivo imaging of the mesenchymal leukemic niche in NOD/SCID mice. (A) Wright-Giemsa stain: Paraffin-embedded MSC-coated scaffold (s), harvested 1 week after in vivo subcutaneous implantation shows vascularization. (B) Control non-coated scaffold is CD45 negative with presence of reticular fibers only. No BM elements are present and AML cells are not retained (C) In vivo implanted MSC-coated scaffold shows presence of adipocytes, blood vessels and nests of AML cells, suggestive of an ectopic human bone marrow environment. (C1-2-3) Intravascular presence of AML cells with one cell migrating in (or out) the vascular space. (C4) Wright-Giemsa stain of multinucleated osteoclast in MSC-coated scaffold. (D) DAB Peroxidase stain for human CD45:CD45 positive myeloid cells (brown) reside in the perivascular stroma in the MSC-scaffold niche (arrows), 1 week after retroorbital AML injection.

FIG. 5: In vitro mesenchymal leukemic niche formation is regulated by SDF-1/CXCL12. Left panel: Phase contrast microscopic imaging of the MSC niche with cobblestone area formation in presence of SDF-1/CXCL12 (10 ng/ml). Right panel: In presence of AMD3100 (10 mM), MSC remain empty and hematopoietic progenitors do not migrate underneath the MSC (see arrows).

FIG. 6: In vivo mesenchymal leukemic niche formation is regulated by SDF-1/CXCL12. AML cells are stained with DAB peroxidase for human CD45 (brown) 4 weeks after injection in MSC-coated scaffolds in NOD/SCID mice. Left panel: SDF-1/CXCL12-treated scaffolds (10 ng/ml) show proliferation of the MSC stromal layer with multiple adherent AML cells. Middle panel: In the AMD3100-treated scaffolds (10 mM) the stromal lining is thin and disrupted at several points, leaving AML cells free floating in proximity. Right panel: The PBS-treated control-scaffold shows a thin single cell MSC stromal layer without disruption, with only a few AML cells attached.

FIG. 7: Leukemia progression in the MSC niche scaffold in vivo. (A-B) DAB peroxidase stain for human CD45 (brown) at 1 week and 4 weeks shows nests of AML cells. Cell-to-cell interaction between AML and MSC is imaged. (C) Wright-Giemsa stain at 8 weeks shows leukemia progression taking over the complete niche and invading other neighboring niche space (arrows). (D) None-coated negative control scaffold shows presence of reticular fibers.

FIG. 8: Ki-67 stain of AML in the MSC niche scaffold in vivo. (A-B-C) AML stain positive for cytoplasmic marker Ki67 (orange) as they invade from one niche to another (arrow). (D-E-F) Non-adherent AML cells are Ki67 positive, while non-adherent AML cells remain Ki67 negative.

### DETAILED DESCRIPTION OF THE INVENTION

As discussed above, the present invention is directed to an in vitro cultured human permissive niche, or human bone marrow microenvironment, comprising a scaffold coated with human mesenchymal stem cells and a culture medium, wherein the stem cells are viable and proliferate in culture and the niche is permissive for the establishment of introduced hematopoietic or leukemic cell populations.

In accordance with the present invention, a scaffold is a three dimensional structure that serves as a suitable support for the grown and proliferation of the stem cells, does not interfere with stem cell growth and viability, and permits adherence of the human mesenchymal stem cells. In the preferred embodiment, the scaffold is an elastomeric matrix that is preferably porous, and more preferably is reticulated and resiliently-compressible. Suitable scaffolds for use in present invention are described in Dalta, et al., U.S. Publication No. 2005/0043585, Brady, et al, U.S. Pat. No. 6,177,522 and Brady, et al., U.S. Publication No. 2002/0142413, which are hereby incorporated by reference. In this regard, the matrix can be made from a thermoplastic elastomer such as polycarbonate polyurethanes, polyether polyurethanes, polysiloxane polyurethanes, hydrocarbon polyurethanes, polyurethanes with mixed soft segments, and mixtures thereof, and preferably is made from polycarbonate polyurethane. It is also within the confines of the present invention that the matrix can be coated with a coating material such as collagen, fibronectin, elastin, hyaluronic acid or mixtures thereof to facilitate cellular ingrowth and proliferation.

With respect to the human mesenchymal stem cells, the stem cells can be normal, aberrant, or oncogenially transformed, and preferably are normal. The stem cells are preferably obtained as bone marrow samples from one or more subjects in accordance with known procedures. However, it is within the confines of the present invention that the mesenchymal stem cells can be isolated from other tissues including cord blood, peripheral blood, fallopian tube, and fetal liver and lung. For purposes of niche analysis, the stem cells may be obtained from a patient suspected of or having leukemia, a patient who has undergone or is undergoing treatment for leukemia, or a patient who is believed to be in remission. The stems cells can be isolated and/or concentrated from the bone marrow samples in accordance with known procedures such as by Ficoll-density gradient separation. The mesenchymal stem cells also may be characterized by one or more markers. In this regard, the mesenchymal stem cells for use in the present invention may be negative for hematopoietic (CD34, CD45) markers and endothelial (CD11b, CD14 and CD31) lineage associated markers, and/or positive for CD29, CD44, CD73, CD105, CD106 and CD166. With respect to phenotypic markers, the mesenchymal stem cells for use in the present invention may be positive for CD90, CD105 and CD146. In another embodiment, a fraction of the mesenchymal stem cells for use in the present invention may express CXCR4. In a preferred embodiment of the present invention, a fraction of the mesenchymal stem cells comprises a rapidly replicating subpopulation of mesenchymal stem cells.

The stem cells in the presence of the scaffold are cultured in a culture medium that supports the growth, proliferation and viability of the stem cells in culture. Preferably, the culture medium includes the chemokine stromal-cell derived factor (SKF-1/CXCL12) available from R&D Systems. The scaffold and stem cells may also be cultured in a suitable cloning cylinder under suitable conditions (e.g., at 37° C., 5%CO2, in humidified air) for a suitable period of time (e.g., 1-5 days). It is also within the confines of the present invention that the scaffolds can be washed to remove any non-adherent cells (e.g., after 1-2 days of culture), followed by a replacement with fresh medium as needed.

The microenvironment can also include myeloid or lymphoid lineage-derived cells, which are desirably added to the microenvironment after establishment of viable and proliferating stem cells on the scaffold. Preferably, the myeloid or lymphoid lineage-derived cells are human, acute or chronic myeloid leukemia cells, human, acute or chronic lymphoid leukemia cells, or human dendritic histiocytic cells. Most preferably, the myeloid lineage-derived cells are human acute myeloid leukemia cells. However, it is within the confines of the present invention that the leukemia cells can be introduced or added to the microenvironment concurrently with the addition or the stem cells or before establishment of a viable and proliferating stem cell culture. The human leukemia cells can be obtained from one subject or a collection of subjects using known procedures. Preferably, the leukemia cells are human acute myeloid leukemia cells. The human acute myeloid leukemia cells can be characterized by one or more markers. In one embodiment, the human acute myeloid leukemia cells are CD45 positive. In another embodiment, the human acute myeloid leukemia cells before introduction into the scaffold are CD34 and CD38 negative. In yet another embodiment, a portion of the human acute myeloid leukemia cells express CXCR4. In an additional embodiment, the acute myeloid leukemia cells are Ki-67 positive or Ki-67 negative.

In accordance with the present invention, the microenvironment can be used to evaluate an agent for anti-leukemic properties. The tested anti-leukemic agent can be targeted against the leukemic cells and/or to the mesenchymal stem cells or the bone marrow microenvironment. In this regard, such a method would include the steps of obtaining or preparing an in vitro human bone marrow microenvironment comprising a scaffold coated with human mesenchymal stem cells (again, preferably after viable and proliferating stem cells are established), introducing to the scaffold human leukemia cells, introducing an agent of interest to the microenvironment containing the leukemia cells, and evaluating the anti-leukemic properties of the agent. For purposes of general drug screening, the stem cells and/or the leukemia cells can be from the same subjects or a collection of subjects, and the microenvironment can be used to identify agents as putative or potential anti-leukemic agents. For purposes of niche analysis, the leukemia cells and the stem cells may be obtained from a single patient having primary or secondary leukemia at the time of diagnosis or at the time of relapse, or a patient who has undergone or is undergoing treatment for leukemia. The leukemic cells may be obtained from the bone marrow, the peripheral blood or a leukapheresis harvest after informed consent from the patient. The microenvironment can then be used to evaluate the therapeutic efficacy of an agent or combination of agents to determine the treatment regimen for that specific patient. For example, if the patient is undergoing treatment with an anti-leukemic agent, the microenvironment can be used to assess whether the patient should continue treatment with the same agent or whether the leukemia is resistant to the agent, and alternative therapy should be employed. Alternatively, the microenvironment can be used to determine the best course of treatment for a patient diagnosed with leukemia by analyzing one or more agents to determine which agent or combination of agents would be therapeutically effective to treat leukemia in the patient. For both general drug screening and niche analysis, an appropriate control can be used. Preferably, the control would include a control microenvironment comprising a scaffold coated with human mesenchymal stem cells, wherein the control microenvironment does not include leukemia cells.

The present invention is also directed to establishment of a permissive niche in a non-human animal comprising a scaffold coated with human mesenchymal stem cells introduced into the animal ectopically, wherein the niche and the model are permissive for the establishment of introduced hematopoietic or leukemic cell populations. Preferably, the non-human animal is a mouse. In accordance with the present invention, the non-human animal is preferably immunosuppressed or immunocompromised, and most preferably is a NOD/SCID mouse. The non-human animal is prepared by culturing a scaffold with human mesenchymal stem cells under conditions permitting the stem cells to coat the scaffold, and introducing the scaffold coated with the human mesenchymal stem cells into the non-human animal ectopically. The characteristics of the scaffold, the stems cells, and the manufacture of the scaffold with the stem cells are as discussed herein. Preferably, the scaffold coated with human mesenchymal stem cells is introduced subcutaneously, and more preferably, is introduced subcutaneously on the back of the non-human animal. It is preferred that a portion of the mesenchymal stem cells survive at least one week after introduction of the scaffold coated with human mesenchymal stem cells into the non-human animal. In addition, it is preferred that the scaffold reveals vascularization one week after introduction of the scaffold coated with human mesenchymal stem cells into the non-human animal. Furthermore, after introduction of the scaffold coated with human mesenchymal stem cells into the non-human animal, the scaffold preferably comprises adipocytes, blood vessels and osteoclasts. For purposes of further study, leukemia cells can be introduced into the non-human animal under conditions permitting the leukemia cells to migrate to the scaffold. The leukemia cells may be from a single patient or a collection of patients, and for purposes of niche analysis, the leukemia cells are desirably from a single patient. In the preferred embodiment, the leukemia cells are human acute myeloid leukemia cells, and may have the characteristics set described herein.

In accordance with the present invention, the non-human animal can be used to evaluate an agent for anti-leukemic properties, targeted against the leukemic cells and/or the mesenchymal stem cells or the bone marrow microenvironment. In this regard, such a method would include the steps of obtaining or preparing an a non-human animal comprising a scaffold coated with human mesenchymal stem cells (again, preferably after viable and proliferating stem cells are established), introducing an agent of interest to the non-human animal, and evaluating the anti-leukemic properties of the agent. It also within the confines of the present invention that the following introduction of scaffold coated with stem cells into the animal, and preferably after the formation of adipocytes, blood vessels and osteoclasts, the scaffold can be removed and tested with the agent in vitro. The non-human animal model can be used for drug screening and niche analysis as described above.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

Introduction

Currently, the hematopoietic niche is defined by two types of engineering cells: osteoblasts (located near the endosteal zone) and endothelial cells (near the vascular sinusoids) [3, 4]. We now defined a third type of cells involved in stem cell niche design i.e. mesenchymal stem cells (located in the perivascular space of the bone marrow). MSC have been studied extensively for their role in tissue repair throughout the body [18], but their proposed function in the BM stem cell niche is a novel concept. In this study, phenotypic markers of lymphoid (CD90), endothelial (CD105) and osteoblast (CD146) lineage have been identified to be present on MSC in the BM. A small subfraction of these particular MSC are also CXCR4 positive [19]. High expression of CXC chemokine ligand 4 (CXCR4) by leukemic blasts and activation of the CXCR4-SDF-1/CXCL12 axis is involved in leukemia progression and disruption of normal hematopoiesis [10]. In addition, a mesenchymal stem cell niche was created in a tissue-engineered construct using a three-dimensional scaffold in combination with stem cells, and was shown to support malignant hematopoiesis within the stromal microenvironment.

The NOD/SCID mice repopulation assay is the current model to assess clonogenicity of human leukemic stem cells. However, one of the major restrictions of the xenotransplant NOD/SCID assay is that the human acute myeloid leukemia (AML) cells need to engraft in a murine bone marrow environment. This hurdle was bypassed by subcutaneous implantation of human mesenchymal stem cell-coated scaffolds in NOD/SCID mice, creating an ectopic permissive human microenvironment for homing and growth of human leukemia. The use of AMD3100 (a CXCR4 antagonist) was tested in this model, and the drug appeared to interact with AML homing by stem cell niche disruption at the level of the stromal layer.

MSC is proposed to provide specific niches in the BM that support survival of leukemic stem cells through signaling via the SDF-1/CXCR4 axis. Synthetic niches have been used in vivo to serve as scaffolding for formation of new tissue [20, 21]. Thus, an in vitro and in vivo bioengineered tissue-model has been developed herein that creates a human BM microenvironment to study the mesenchymal stem cell niche and its interaction with AML. These approaches are useful to build predictive models for drug screen and drug resistance, as well as potential therapeutic targeted drug screening in combination with conventional chemotherapy.

Materials and Methods

Phenotypic Characterization of Mesenchymal Stem Cells

Fresh human BM samples were obtained from orthopedic surgery after informed consent (Tissue Donor Program of the Feinstein Institute). The buffy coat containing the mononucleated cells (MNC) was isolated by Ficoll-density gradient separation (Stem Cell Technology, Vancouver). MNC were plated at low density in 6-well plates in Alpha-Mem (Lonza), 20% heat-inactivated fetal bovine serum (Hyclone), 5 mM L-Glutamine and 100 Units/ml Peni/Strep (Stem Cell Technologies) for overnight adherence. After 24-48 hours, the non-adherent cell fraction was discharged by rigorous pipetting. At day 5, single-cell derived mesenchymal colonies were processed for immunohistochemistry (Vectastain ABC kit, Vector Laboratories, Burlingame, Calif.). MSC were stained for CD90/Thy-1 (Mouse anti-human mAb, Clone 5E10, BD Pharmingen), CD105 (Mouse anti-human mAb, clone 266, BD Pharmingen), CD146 (Mouse monoclonal [P1H12] Abcam). Cells were also labeled for flow cytometry with CD34-APC (Miltenyi Biotec), CD105-FITC (R&D), CD45-FITC, CD90-PECy5, CD146-PE and CXCR4-PE (BD Pharmingen) including matching isotype controls.

Phenotypic Characterization of Acute Myeloid Leukemia Cells

Primary AML cells were obtained from anonymous donors by clinically indicated leukapheresis harvest at the time of diagnosis. The sample was processed and MNC fraction was cryopreserved in aliquots with 10% DMSO. Thawed cells were washed and inoculated on MSC colonies on day 5 of MSC tissue culture or directly injected into subcutaneously implanted scaffold in NOD/SCID mice (see below). AML cells were labeled for flow cytometry and cell sorting with CD34-APC (Miltenyi Biotec), CD38-PECy7 (eBiosciences), CD45-FITC and CXCR4-PE (BDPharmingen). AML cells were incubated according to manufacturer's protocol with Hoechst stain (Molecular Probes-Invitrogen) and Pyronin Y (Sigma-Aldrich) for quantitive DNA and RNA measurement respectively during cell cycle analysis on a FACS laser instrument (Becton Dickinson).

Preparation of MSC-Coated Scaffolds for Mesenchymal Stem Cell Niche Analysis

In vitro Analysis

Polyurethane scaffold test discs (10×1.5 mm—Biomerix) were placed inside a cloning cylinder (Millipore Corporation, MA), one cylinder in each well of a 24-well plate (Falcon, BD). Inserted scaffolds were seeded with freshly harvested human BM-derived MSC (1×10⁷ cells/disc,) and cultured in alpha-mem/20% FBS with SDF-1/CXCL12 (10 ng/ml, R&D Systems) or in presence of AMD3100 (10 μM, Sigma-Aldrich) at 37° C., 5% CO₂ in humidified air. After 24 hours, the scaffolds were flushed rigorously with PBS to remove any non-adherent cells and fed with fresh medium for continued tissue culture for 5 days. At day 5, the scaffolds were inoculated with normal CD34+ HSC (1×10⁵—Stem Cell Technology, Vancouver) or primary AML cells (1×10⁷—cryopreserved and thawed). After 1 week, the scaffolds were fixed, paraffin-embedded and stained for histological analysis. Imaging was done by inverted microscopy (Zeiss-Axiovert) and photographed by a Nikon digital camera.

In vivo Analysis

Polyurethane scaffolds (10×1.5 mm—Biomerix), coated in vitro with human BM-derived MSC (day 5-7), were implanted in a subcutaneous pocket on the dorsum of non-irradiated NOD/SCID mice (Jackson laboratory) according to an approved IACUC animal protocol. Empty scaffolds (without MSC seeding) were used as negative controls. CD34+ HSC (1×10⁵—Stem Cell Technology, Vancouver) or primary AML cells (1×10⁷—cryopreserved and thawed) were injected either in situ or retro-orbital in the mice and analyzed for engraftment. The mice were treated twice per week with in situ injections of SDF-1/CXCL12 (10 ng/ml), AMD3100 (10 μM) or PBS (control). At week 1, week 4 and week 8, mice were sacrificed and the scaffolds, femurs and spleens were processed and evaluated for cell survival in the mesenchymal niche by immunohistochemistry.

Immunohistochemistry on Scaffolds

Harvested scaffolds were fixed in formalin solution 10%, neutral buffered (HistoPak-StatLab Medical Products-TX) and paraffin embedded and cut on slides. After initial deparaffinization, the slides are subject to eight minutes antigen retrieval (citrate buffer pH 6.6) and then incubated with the primary antibody for 32 minutes (Benchmark XT automated stainers—mouse monoclonal anti-CD45 antibody (Ventana Clone RP2/18) or anti-Ki-67 rabbit monoclonal primary antibody (Ventana). Negative control stains without antibody presence, were performed to rule out none-specific staining.

Electron Microscopy

MSC-coated scaffolds were fixed by immersion in 2%, 0.05M cacodylate buffered, glutaraldehyde, post-fixed in OsO4, dehydrated in a graded series of ethanol and prepared for electron microscopic study by standard methods. Appropriate cellular areas were identified on one micron plastic sections by light microscopy and their ultra structure was evaluated using a JEOL JEM 100CXII transmission electron microscope.

Results

Phenotypic Identification

Mesenchymal Stem Cell Phenotype

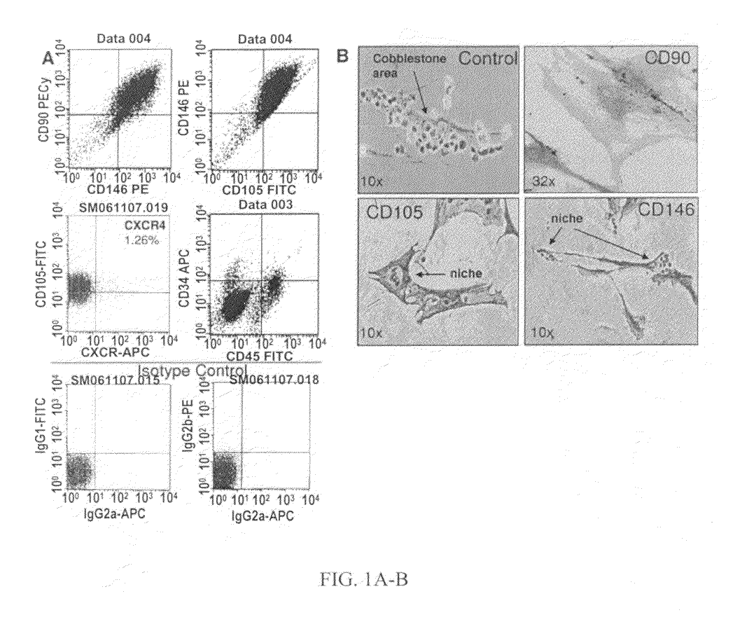

So far, MSC have had a diversity of characterization that can be explained by their tissue of origin (BM, cord blood, fat tissue, bone spicules etc.), isolation methods and culture conditions [22]. In general terms, MSC are negative for hematopoietic (CD34, CD45) or endothelial (CD11b, CD14, CD31) lineage associated markers, but stain positive for CD29, CD44, CD73, CD105, CD106 and CD166 [17, 23]. Phenotypic markers CD90, CD105 and CD146 are present on the MSC that contribute to the niche architecture. In addition, a small subtraction of MSC (1.26%) was found to express CXCR4 by flowcytometry (FIG. 1A). Plated at a very low density, MSC illustrate the typical cobblestone appearance by phase contrast imaging, suggesting support of active hematopoiesis in a two-dimensional tissue culture. Nests of HSC could be found sculpted in the mesenchymal cytoplasm on CD90, CD105 and CD146 positive cells by immunohistochemistry (FIG. 1B).

Acute Myeloid Leukemia Phenotype

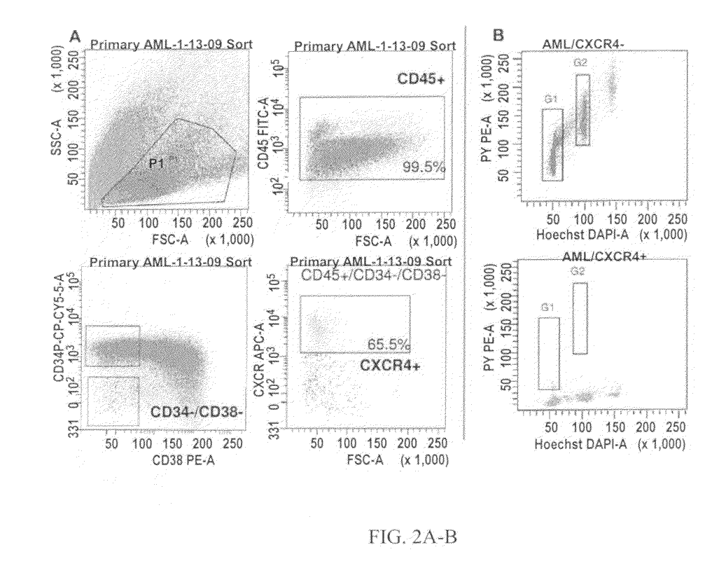

A large number of aliquots of a single AML patient-leukapheresis product were cryopreserved, which allows for reproducibility among different experiments. Primary cells may mimic closely the natural cell biological behavior of leukemia cells in their niche, in contrast to tissue culture-adapted AML cell lines which often become stroma-independent for their growth. The primary AML cells were 99.5% CD45 (leukocyte common antigen) positive. A CD45+/CD34−/CD38− immature AML subpopulation was sorted [24] and 65.5% of these cells expressed CXCR4 [25]. In addition, this CXCR4+ AML subset was in the G0/quiescent phase of the cell cycle (FIG. 2A-B). Quiescence of HSC is critical for stem cell pool maintenance and CXCR4 is required for the quiescence of primitive normal hematopoietic cells to sustain normal hematopoiesis [26]. These data support the notion that CXCR4 expression is a defining characteristic of the leukemic stem cell, in parallel with findings in normal hematopoietic stem cells [10, 26].

Morphological Identification

Traditionally in early passage cultures, 2 distinct kinds of MSC have been defined by their growth rate: rapidly self-replicating spindle-shaped cells (RS-cells) predominate in the first few days after plating the cells at low density (50 cells/cm2), followed by broader, slowly replicating cells (SR-cells) that predominate as cultures become confluent. At much later times in culture after multiple passages, very large and mature MSC appear [27, 28]. Although these three morphologically distinct MSC cell types have been observed, no correlation has been made so far between the time-based morphology and the practical role of MSC to contribute to stem cell niche formation. Based on their biological functional characteristics, at day 1-5 in culture a rapid-replicating MSC (RS-MSC) with a broad very thin cytoplasm was identified. RS-MSC attract hematopoietic progenitors or AML cells that migrate underneath RS-MSC, forming the traditional cobblestone pattern of hematopoiesis (FIG. 1B). RS-MSC are a very rare population with a frequency of 0.001% and their functional characteristics that contribute to the niche neighborhood are lost by subsequent passage of the cells.

Mesenchymal Stem Cell Niche Interactions In Vitro

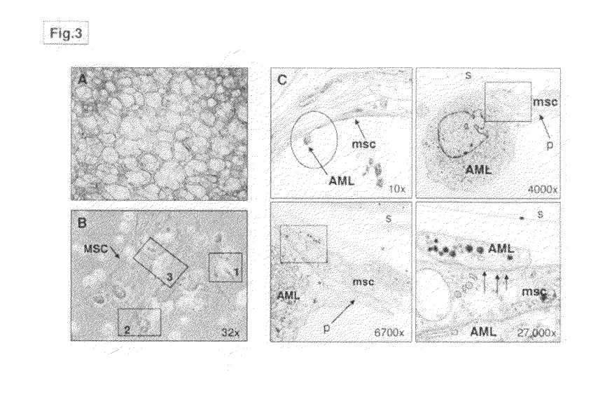

The ‘gold standard’ for cell biological imaging has been tissue culture. However, the two-dimensional structure of tissue culture limits the observation of cell-to-cell interactions that happen in real-time live tissues. A spatial distribution of cells within a three-dimensional matrix is critical to mimic the complex cellular organization of the BM microenvironment, and for retention of the cells at the intended site. Long-term 3D tissue culture of leukemic bone marrow primary cells in a biomimetic osteoblast niche has been described [29]. The data presented differ from our study at several points. Bio-derived bone is used as a scaffold in the osteoblast niche and the MSC are differentiated into osteoblast by use of osteogenic medium. The MSC utilized in the osteoblast niche assay are harvested at passage 3-5. An early passage MSC was used to maintain multipotentiality of the MSC to be induced into a full bone marrow environment. MSC at later passages are more lineage restricted with loss of mesenchymal stem cell niche function. The goal was to fabricate a tissue-engineered construct using a polyurethane three-dimensional scaffold (10 mm diameter, 1.5 mm thickness-Biomerix) (FIG. 3A), in combination with MSC derived from normal human BM to investigate its potential to support malignant hematopoiesis within a stromal microenvironment in vitro and in vivo.

First, the scaffold was coated with MSC in the presence of SDF-1/CXCL12. Initial results were encouraging and revealed not only adhesion of the MSC to the scaffold but also cell division, implying survival and proliferation. After successful MSC-coating, the scaffolds were inoculated with AML cells for ultrastructural analysis. Others have described that within the osteoblast niche of the BM, HSC adhere to BM osteoblasts by developing long, tentacle-like projections, called uropods [30]. In two-dimensional culture, AML cells attach, migrate and form pseudo-uropods underneath the MSC (FIG. 3B). Similarly, cell-to-cell (AML/MSC) interactions could be observed at a single cell level in the MSC-coated 3D-scaffold (FIG. 3C-FIG. 7) and AML cells developed pseudo-uropods that anchor intimately to MSC, as illustrated by electron microscopy (FIG. 3C). After 1 week, the MSC-coated scaffold retained in vitro the presence of AML cells. The non-coated control scaffold remained empty, confirming the importance of MSC for AML cell retention within the niche.

Mesenchymal Stem Cell Niche Interactions In Vivo in NOD/SCID Mice

The nonobese diabetic/severe combined immunodeficient (NOD/SCID) mice assay is the current model for assessment of human normal and leukemic stem cells. However, about 50% of the AML patient samples are unable to initiate leukemia in NOD/SCID mice. This has been attributed to an important difference in cell biological behavior between leukemic initiating cells of engrafting and non-engrafting AML cases that correlates with treatment response [31]. SCID-leukemia initiation cells share many properties with normal HSC, namely phenotype, quiescence and in vitro CXCR4-mediated migration [32, 33, 34]. Various factors, such as adhesion molecules, cytokines and receptors that affect normal HSC engraftment may be applicable to AML NOD/SCID engraftment as well. The one factor that has not been studied extensively (due to the limitation of a functional assay and the complexity of the in vivo BM environment) is the role of the mesenchymal compartment in engraftment. One of the major restrictions of the xenotransplant NOD/SCID assay is that human AML cells are expected to engraft in a murine BM environment. Other limitations of conventional cell infusions or injections for xenotransplantation include poor delivery and poor retention of cells at the intended site or cell death due to loss of anchorage (anoikis) [20]. The goal was to create an ectopic mesenchymal stem cell niche in NOD/SCID mice as a permissive human microenvironment for homing and growth of human leukemia.

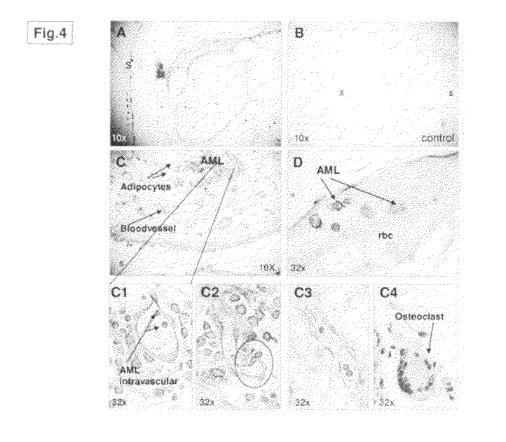

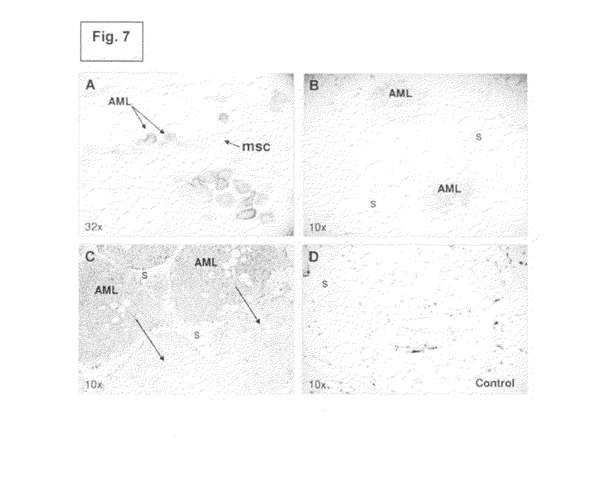

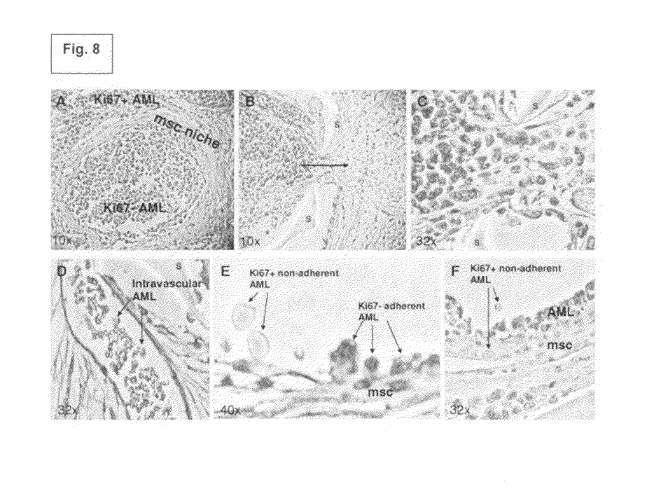

First, it was asked whether the MSC-coated scaffold supports MSC cell survival in vivo. Not only did the MSC survive in NOD/SCID mice, but 1 week after subcutaneous implantation the scaffolds revealed vascularization, while the non-coated empty control scaffold had only growth of reticular fibers with signs of murine foreign body reaction at the borders of the scaffold. (FIG. 4A-B). Eight weeks later, the scaffold showed the presence of adipocytes, blood vessels and osteoclasts, suggestive of an ectopic human BM environment (FIG. 4C1-2-3-4). All scaffolds were well tolerated in the immunodeficient hosts, without infection or ulceration. Second, it was asked if the ectopic human BM environment could be supportive of human hematopoiesis. Human CD45-positive myeloid cells resided in the perivascular space of the scaffold stroma, 1 week after retroorbital or in situ injection. AML cells were scattered throughout the niche and present in proximity to the blood vessels (FIG. 4D). However, not only did the AML cells home and survive, but at 8 weeks Ki-67 positive AML cells took over the whole niche space and invaded from one niche site to another. Ki67 is a histochemical cytoplasmic marker for active mitosis. The AML cells adherent to MSC remained Ki67 negative while non-adherent AML cells stain positive for Ki67, supporting the idea that the MSC niche provides a protective milieu for dormant AML cells. In the empty control scaffold, no bone marrow elements developed and AML cells did not survive (FIGS. 7 and 8). Murine femurs and spleens were negative for human AML cells by immunohistochemistry.

Biological Function of the Mesenchymal Stem Cell Niche In Vitro

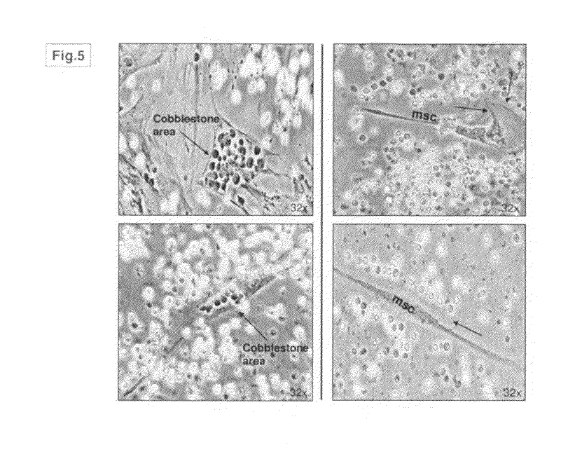

MSC was identified as niche-maker cells, and the crucial role of the SDF1(CXCL12)/CXCR4 axis in vitro was then investigated. In the presence of SDF-1/CXCL12, phase contrast imaging illustrated cobblestone formation. The hematopoietic progenitors became phase-contrast negative as they migrated underneath the thin cytoplasm of the MSC. In contrast, the cytoplasm remained empty in the presence of AMD3100 (a CXCR4 antagonist), suggestive of niche-disruption (FIG. 5).

Biological Function of the Mesenchymal Stem Cell Niche In Vivo

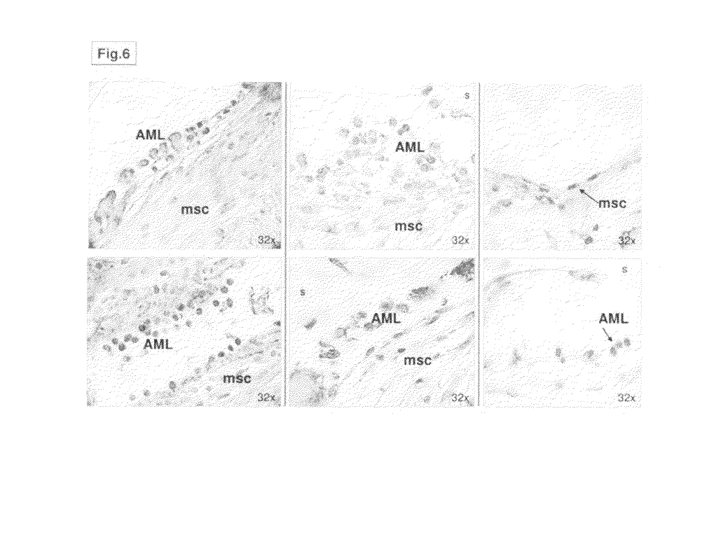

The interaction between the chemokine SDF-1/CXCL12 and its receptor CXCR4 plays a major role in leukemogenesis and leukemia progression [10, 35]. Antibody blocking studies revealed that engraftment of normal human HSC and repopulation in NOD/SCID mice is dependent on the interaction between CXCR4 and SDF-1/CXCL12 [7]. In AML, CXCR4 also regulates migration of transplanted human leukemia in NOD/SCID mice. However, the exact mechanism of AML cell engraftment in NOD/SCID mice via the SDF-1(CXCL12)/CXCR4 axis is not fully understood, since CXCR4 expression on AML blasts is highly variable [36] and the stromal niche cell involved has not been identified so far. The role of the SDF-1(CXCL12)/CXCR4 axis in the mesenchymal stem cell niche in vivo was then established. Scaffold-implanted mice were divided in two treatment arms: one arm received SDF-1/CXCL12 (10 ng/ml) by biweekly in situ injection in the MSC-coated scaffold and the other arm received AMD3100 (10 □M), a CXCR4 antagonist. Control mice received PBS buffer only. Four weeks later, the SDF-1/CXCL12-treated scaffolds showed thick proliferation of the MSC stromal layer with multiple adherent AML cells present, while the AMD3100-treated scaffold had a thin stromal lining that was disrupted at several points, leaving AML cells free floating in proximity. The PBS-control scaffold showed a single layer of MSC with only a few AML cells attached (FIG. 6).

Each experiment was performed in duplicate, with two mice per experiment in each treatment arm and one mouse as a negative control. Multiple slides per scaffold were analyzed for imaging by immunohistochemistry and light microscopy. Negative controls include empty scaffold (no MSC or AML), PBS-injected scaffold (no SDF-1/CXCL12 or AMD3100) and immunohistochemistry without antibody presence.

Discussion

In stem cell biology, there is an emerging trend to understand the different niche-players and their functional interaction. Two candidate BM niches have been named as the vascular niche and the endosteal niche. The endosteal niche has been delineated by the physical localization of hematopoietic progenitor cells close to osteoblasts in the endosteum of the bone [5]. The osteoblastic niche provides signaling for the maintenance of the repopulating cells in an undifferentiated state [3, 37]. SDF-1/CXCL12 is not only a major chemoattractant for HSC retention but also a regulatory factor that controls quiescence of primitive hematopoietic cells [26]. CXCR4 antagonists disrupt the endosteal niche and result in rapid mobilization of HSC. Whereas quiescent cells favor the dormant surroundings of the endosteal niche, the vascular niche attracts cells for differentiation and maturation before exiting the BM microenvironment into the peripheral circulation [4]. The common denominator for both niches is a population of reticular cells that abundantly expressed SDF-1/CXCL12 named CXCL12-abundant reticular (CAR) cells. CAR cells have not been fully characterized, nor has their cellular source been indentified [38].

Characteristics of the Mesenchymal Stem Cell Niche

Defining MSC in vitro is complicated because of they are easily induced to differentiate in tissue culture. Extrapolating MSC in vitro data to the complexity of the BM environment has been hampered by lack of a functional in vivo study model that allows imaging of their anatomic location as well as their biological interaction with HSC. The identification of the MSC niche is necessary to validate results obtained in vitro and to elucidate the physiological functions of these adult stem cells. However, the absence of MSC specific markers and their modification in cultures hinder MSC identification in vitro and in vivo [22].

MSC are usually defined by their capacity to differentiate into at least one mature cell type. We identified phenotypic markers of MSC coinciding with their in vitro operational behavior i.e. the formation of protective niches by cobblestone formation. Cultures of human MSC are morphologically heterogeneous and the cells undergo delicate changes as they mature with subsequent passages, resulting in loss of multipotentiality. Extensive antibodies screens by several investigators have not discovered any that discriminate RS-MSC from the later more differentiated and mature MSC [26]. MSC were distinguished by their cell biological behavior, and showed that cobblestone forming-MSC at day 5 in culture, express not only the pericyte marker CD146, but in addition express the vascular endothelial marker CD105 and the hematopoietic marker CD90 (Thy-1). MSC are negative for the hematopoietic lineage markers CD45 and CD34, but a small subfraction does express CXCR4. These marker studies are in accordance with the criteria proposed by the Mesenchymal and Tissue Stem Cell Committee of the International Society for Cellular Therapy to define human MSC [39]. If the culture were maintained longer than 5 days, the cell surface markers were down regulated. Prior to day 5, rapid-cycling MSC transitioned into slow-dividing more mature MSC and the traditional fibroblast colonies appeared, no longer supportive of cobblestone formation. These findings suggest that the adult BM contains a rare (0.001%) subpopulation of very primitive and undifferentiated MSC that attracts hematopoietic progenitors in culture.

Much data have been generated defining the HSC niche in murine models, including the effect of micro-environment-specific defects and their impact on mobilization of HSC [39]. Intramedullary transplantation of enhanced green fluorescent protein-marked human MSC (eGFP-MSC) into NOD/SCID mice resulted in a functional human hematopoietic microenvironment integrated in the BM of the murine host. eGFP-MSC differentiated into myofibroblasts, BM stroma cells, osteocytes in bone, bone-lining osteoblasts, and endothelial cells [40]. A recent study reported that BM-derived CD146+ reticular cells can create a hematopoietic microenvironment in heterotopic sites when transplanted subcutaneously in a xenograft transplantation model [41]. However, the recommended 16-26 weeks posttransplant required for analysis of the HSC repopulation assay impedes fast progress in the field and is very costly. Alternative means of assessing engraftment of HSC are clearly needed but in vivo data to regenerate a human equivalent bone marrow microenvironment have been limited.

In this study, a human MSC-derived ectopic BM microenvironment was created on the back of NOD/SCID mice to study the biological interaction of leukemic hematopoietic stem cells in the MSC niche. A variety of naturally derived materials and synthetic polymers are currently in development as vehicles for stem cell transplantation because of their ability to provide adhesion for interacting cells [20]. In this study, a three-dimensional scaffold provided the supportive network for a bioengineered tissue-model after coating with MSC from human BM samples. The MSC-coated scaffold revealed presence of adipocytes, osteoclast and blood vessels, mimicking a human BM microenvironment that supports growth of inoculated human myeloid leukemia. The control empty scaffold without human elements, showed only the ingrowths of murine-derived reticular fibrous tissue. These data suggest that the scaffolds support ectopic human microenvironment formation derived from primitive MSC. In addition, this model circumvents the limitations of conventional cell infusions or injections including poor delivery and poor retention of cells at the intended site. This allows for faster engraftment and early analysis (week 4-8) of repopulation in the scaffold, saving time and money.

The leukemic Mesenchymal Stem Cell Niche

AML accounts for approximately 15% of all childhood leukemia. 25% of the patients relapse during or after treatment due to MRD. The influence of the microenvironment on leukemia chemosensitivity has not been fully outlined. It was hypothesized that MSC provide specific niches for leukemic stem cells through signaling via SDF-1(CXCL12)/CXCR4 axis. Regulation of the passage of leukemic stem cells in and out their niche by cell cycles manipulation and modification of the niche could be proven a potential strategy for treatment of chemoresistance and disease eradication in childhood AML. Clinically, AML is a disease with a broad spectrum of presentation due to the hierarchical structure within AML subtypes. Based on clinical observations, AML blasts have been divided in an immature CD34+/CD38− fraction and a more mature CD34+/CD38+ one [33]. Only the immature CD34+/CD38− fraction seem to contain SCID leukemia-initiating cells after xenotransplant, in analogy with SCID-repopulating cells in normal human hematopoiesis in NOD/SCID mice. We isolated primary AML cells that were CD45+/CD34−/CD38− and 65% of this immature population expresses CXCR4. Moreover, the immature CXCR4-expressing AML population appears to be in the quiescent phase of the cell cycle. In addition, Ki-67 (a marker for cell division) immunohistochemical staining was present with leukemia disease progression in the implanted scaffolds. But AML cells adherent to MSC remain Ki-67-negative, while those that are non-adherent or reside intravascular show mitotic activity as indicated by Ki-67 stain (FIG. 8). These findings support the hypothesis of the present invention, that AML cells “hiding” in the mesenchymal stem cell niche are quiescent, leading to chemoresistance.

In normal hematopoiesis, human stem cell engraftment is dependent on CXCR4 and thus CXCR4 antagonists caused rapid mobilization of human CD34+ cells. Elevated CXCR4 levels have been described in AML and predict poor prognosis [32] and targeting CXCR4 with its antagonist AMD3465 has been shown to prevent the chemoprotective effects of the stromal cell-leukemia interaction [42]. However, a careful in vivo validation model that provides insight into the cellular biology of the niche has not been described. The human allograft model hosted in NOD/SCID mice presented in this study does indeed serve this purpose. It was indeed showed that SDF-1/CXCL12 upregulates adhesion of AML cells to the stroma, but that SDF-1/CXCL12 also induces hyperproliferation of the mesenchymal stroma compartment. In contrast, in the presence of AMD3100, the MSC stroma became ruffled and non-adherent and AML remained only loosely attached.

Conclusions

If the endosteal niche induces HSC dormancy, and the vascular niche proliferation and differentiation, so what is then the function of the mesenchymal niche? In tissue repair, MSC are quickly mobilized to the place of injury to perform ‘first aid’ repair and regeneration of the injured tissue. An interesting hypothesis for future exploration is that the MSC niche in the BM functions as the repair station for vulnerable HSC while they transition from their dormant cell cycle at the endosteal niche towards the highly proliferative phase necessary for differentiation at the vascular niche. It is obvious that in this function, the physical location of MSC in the perivascular space between the endosteum and the vascular sinusoids in the BM is an anatomically optimal way station. If any bone marrow insult occurs, the MSC becomes activated and secretes SDF-1/CXCL12, thereby attracting HSC to a safe haven to protect and repair them from injury. Leukemic stem cells have been shown to downregulate SDF-1/CXCL12 secretion by MSC once the leukemic stem cells occupy the BM niche, thereby regulating spatial competition with normal HSC by reducing their major chemoattractant [43]. In addition, MSC repress immune surveillance, stimulate angiogenesis and provide anti-apoptotic stimuli, all beneficial factors for the leukemic niche hijackers to ensure their survival [44].

This research crosses the interface of bioengineering, cell biology and drug development. The novel assay fills the gap at the junction of basic research and human application to study and gain insights in mechanisms to overcome clinical chemoresistance in AML. Targeted niche disruption in combination with conventional chemotherapy represents an intriguing new approach to overcome chemoresistance that can translate into improved therapeutic outcomes for patients with AML.

List of Cited References

1. L. A. Johnston, Competitive interactions between cells: death, growth, and geography, Science 324, 1679-1682 (2009).

2. R. Schofield, The relationship between the spleen colony-forming cell and the haemopoietic stem cell, Blood Cell 4, 7-25 (1978).

3. J. Zhang, C. Niu, L. Ye, H. Huang, X. He, W. G. Tong, J. Ross, J. Haug, T. Johnson, J. Q. Feng, S. Harris, L. M. Wiedemann, Y. Mishina, and L. Li, Identification of the haematopoietic stem cell niche and control of the niche size, Nature 425, 836-841 (2003).

4. H. G. Kopp, S. T. Avecilla, A. T. Hooper, and S. Rafii, The bone marrow vascular niche: home of HSC differentiation and mobilization, Physiology (Bethesda) 20, 349-356 (2005).

5. R. L. Porter and L. M. Calvi, Communications between bone cells and hematopoietic stem cells, Arch Biochem Biophys 473, 193-200 (2008).

6. T. Matsunaga, N. Takemoto, T. Sato, R. Takimoto, I. Tanaka, A. Fujimi, T. Akiyama, H. Kuroda, Y. Kawano, M. Kobune, J. Kato, Y. Hirayama, S. Sakamaki, K. Kohda, K. Miyake, and Y. Niitsu, Interaction between leukemic-cell VLA-4 and stromal fibronectin is a decisive factor for minimal residual disease of acute myelogenous leukemia, Nat Med 9, 1158-1165 (2003).

7. A. Peled, I. Petit, O. Kollet, M. Magid, T. Ponomaryov, T. Byk, A. Nagler, H. Ben-Hur, A. Many, L. Shultz, O. Lider, R. Alon, D. Zipori, and T. Lapidot, Dependence of human stem cell engraftment and repopulation of NOD/SCID mice on CXCR4, Science 283, 845-848 (1999).

8. J. J. Lataillade, D. Clay, P. Bourin, F. Herodin, C. Dupuy, C. Jasmin, and M. C. LeBousse-Kerdilles, Stromal cell-derived factor 1 regulates primitive hematopoiesis by suppressing apoptosis and by promoting G0/G1 transitioning in CD34+ cells: evidence for an autocrine/paracrine mechanism, Blood 99, 1117-1129 (2002).

9. I. Petit, D. Jin, and S. Rafii, The SDF-1-CXCR4 signaling pathway: a molecular hub modulating neo-angiogenesis, Trends Immunol 28, 299-307 (2007).

10. F. Ayala, R. Dewar, M. Kieran, and R. Kalluri, Contribution of bone microenvironment to leukemogenesis and leukemia progression, Leukemia 3, 1-9 (2009).

11. I. E. Konstantinov, In search of Alexander A. Maximow: the man behind the unitarian theory of hematopoiesis, Perspect Biol Med 43, 269-276 (2000).

12. E. A. McCulloch and J. E. Till, The radiation sensitivity of normal mous bone marrow cells, determined by quantitative marrow transplantation into irradiated mice, Radiat Res 13, 115-125 (1960).

13. A. J. Friedenstein, R. K. Chailakhyan, N. V. Latsinik, A. K. Panasyuk, and I. V. Keiliss-Borol, Stromal cells responsible for transferring the microenvironment of the hematopoietic tissues. Cloning in vitro and retransplantation in vivo, Transplantation 17, 331-340 (1974).

14. T. M. Dexter, T. D. Allen, and L. G. Lajtha, Conditions controlling the proliferation of haemopoietic stem cells in vitro, J Cell Physiol 91, 335-344 (1977).

15. T. M. Dexter, M. A. Moore, and A. P. Sheridan, Maintenance of hemopoietic stem cells and production of differentiated progeny in allogeneic and semiallogeneic bone marrow chimeras in vitro, J Exp Med 145, 1612-1616 (1977).

16. L. Diaz-Flores, R. Gutierrez, J. F. Madrid, H. Varela, F. Valladares, E. Acosta, P. Martin-Vasallo, and L. Diaz-Flores, Jr., Pericytes. Morphofunction, interactions and pathology in a quiescent and activated mesenchymal cell niche, Histol Histopathol 24, 909-969 (2009).

17. M. F. Pittenger, A. M. Mackay, S. C. Beck, R. K. Jaiswal, R. Douglas, J. D. Mosca, M. A. Moorman, D. W. Simonetti, S. Craig, and D. R. Marshak., Multilineage potential of adult mesenchymal stem cells, Science 284, 143-147 (1999).

18. T. S. Stappenbeck and H. Miyoshi, The role of stromal stem cells in tissue regeneration and wound repair, Science 324, 1666-1669 (2009).

19. R. F. Wynn, C. A. Hart, C. Corradi-Perini, L. O'Neill, C. A. Evans, J. E. Wraith, L. J. Fairbairn, and I. Bellantuono, A small proportion of mesenchymal stem cells strongly expresses functionally active CXCR4 receptor capable of promoting migration to bone marrow, Blood 104, 2643-2645 (2004).

20. D. Discher, D. J. Mooney, and P. Zandstra, Growth factors, matrices and forces combine and control stem cells, Science 324, 1673-1677 (2009).

21. S. Z. Yow, C. H. Quek, E. K. Yim, C. T. Lim, and K. W. Leong, Collagen-based fibrous scaffold for spatial organization of encapsulated and seeded human mesenchymal stem cells, Biomaterials 30, 1133-1142 (2009).

22. L. da Silva Meirelles, A. I. Caplan, and N. B. Nardi, In search of the in vivo identity of mesenchymal stem cells, Stem Cells 26, 2287-2299 (2008).

23. G. Chamberlain, J. Fox, B. Ashton, and J. Middleton, Concise review: Mesenchymal Stem Cells: Their phenotype, differentiation capacity, immunological features and potential for homing, Stem Cells 25, 2739-2749 (2007).

24. D. Bonnet and J. E. Dick, Human acute myeloid leukemia is organized as a hierarchy that originates from a primitive hematopoietic cell, Nat Med 3, 730-737 (1997).

25. A. C. Spoo, M. Lubbert, W. G. Wierda, and J. A. Burger, CXCR4 is a prognostic marker in acute myelogenous leukemia, Blood 109, 786-791 (2007).

26. Y. Nie, Y. C. Han, and Y. R. Zou, CXCR4 is required for the quiescence of primitive hematopoietic cells, J Exp Med 205, 777-783 (2008).

27. D. C. Colter, I. Sekiya, and D. J. Prockop, Identification of a subpopulation of rapidly self-renewing and multipotential adult stem cells in colonies of human marrow stem cells, PNAS 98, 7841-7845 (2001).

28. I. Sekiya, B. L. Larson, J. R. SMith, R. Pochampally, J. G. Cui, and D. J. Prockop, Expansion of human adult stem cells from bone marrow stroma: Conditions that maximize the yields of early progenitors and evaluate their quality, Stem Cells 20, 530-541 (2002).

29. L. Hou, T. Liu, J. Tan, W. Meng, L. Deng, H. Yu, X. Zou, and Y. Wang, Long-term culture of leukemic bone marrow primary cells in biomimetic osteoblast niche, Int J Hematol 11, 11 (2009).

30. J. M. Gillette, A. Larochelle, C. E. Dunbar, and J. Lippincott-Schwartz, Intercellular transfer to signalling endosomes regulates an ex vivo bone marrow niche, Nat Cell Biol 11, 303-311 (2009).

31. D. J. Pearce, D. Taussig, K. Zibara, L. L. Smith, C. M. Ridler, C. Preudhomme, B. D. Young, A. Z. Rohatiner, T. A. Lister, and D. Bonnet, AML engraftment in the NOD/SCID assay reflects the outcome of AML: implications for our understanding of the heterogeneity of AML, Blood 107, 1166-1173 (2006).

32. E. J. Rombouts, B. Pavic, B. Lowenberg, and R. E. Ploemacher, Relation between CXCR-4 expression, Flt3 mutations, and unfavorable prognosis of adult acute myeloid leukemia, Blood 104, 550-557 (2004).

33. W. I. Chan and B. J. Huntly, Leukemia stem cells in acute myeloid leukemia, Semin Oncol 35, 326-335 (2008).

34. T. Lapidot, C. Sirard, J. Vormoor, B. Murdoch, T. Hoang, J. Caceres-Cortes, M. Minden, B. Paterson, M. A. Caligiuri, and J. E. Dick, A cell initiating human acute myeloid leukaemia after transplantation into SCID mice, Nature 367, 645-648 (1994).

35. J. A. Burger and T. J. Kipps, CXCR4: a key receptor in the crosstalk between tumor cells and their microenvironment, Blood 107, 1761-1767 (2006).

36. S. Tavor, I. Petit, S. Porozov, A. Avigdor, A. Dar, L. Leider-Trejo, N. Shemtov, V. Deutsch, E. Naparstek, A. Nagler, and T. Lapidot, CXCR4 regulates migration and development of human acute myelogenous leukemia stem cells in transplanted NOD/SCID mice, Cancer Res 64, 2817-2824 (2004).

37. L. M. Calvi, G. B. Adams, K. W. Weibrecht, J. M. Weber, D. P. Olson, M. C. Knight, R. P. Martin, E. Schipani, P. Divieti, F. R. Bringhurst, L. A. Milner, H. M. Kronenberg, and D. T. Scadden, Osteoblastic cells regulate the haematopoietic stem cell niche, Nature 425, 841-846 (2003).

38. T. Sugiyama, H. Kohara, M. Noda, and T. Nagasawa, Maintenance of the hematopoietic stem cell pool by CXCL12-CXCR4 chemokine signaling in bone marrow stromal cell niches, Immunity 25, 977-988 (2006).

39. M. Dominici, K. Le Blanc, I. Mueller, I. Slaper-Cortenbach, F. Marini, D. Krause, R. Deans, A. Keating, D. Prockop, and E. Horwitz, Minimal criteria for defining multipotent mesenchymal stromal cells. The International Society for Cellular Therapy position statement, Cytotherapy 8, 315-317 (2006).

40. Y. Muguruma, T. Yahata, H. Miyatake, T. Sato, T. Uno, J. Itoh, S. Kato, M. Ito, T. Hotta, and K. Ando, Reconstitution of the functional human hematopoietic microenvironment derived from human mesenchymal stem cells in the murine bone marrow compartment, Blood 107, 1878-1887 (2006).

41. B. Sacchetti, A. Funari, S. Michienzi, S. Di Cesare, S. Piersanti, I. Saggio, E. Tagliafico, S. Ferrari, P. G. Robey, M. Riminucci, and P. Bianco, Self-renewing osteoprogenitors in bone marrow sinusoids can organize a hematopoietic microenvironment, Cell 131, 324-336 (2007).

42. Z. Zeng, Y. X. Shi, I. J. Samudio, R. Y. Wang, X. Ling, O. Frolova, M. Levis, J. B. Rubin, R. R. Negrin, E. H. Estey, S. Konoplev, M. Andreeff, and M. Konopleva, Targeting the leukemia microenvironment by CXCR4 inhibition overcomes resistance to kinase inhibitors and chemotherapy in AML, Blood 113, 6215-6224 (2009).

43. A. Colmone, M. Amorim, A. L. Pontier, S. Wang, E. Jablonski, and D. A. Sipkins, Leukemic cells create bone marrow niches that disrupt the behavior of normal hematopoietic progenitor cells, Science 322, 1861-1865 (2008).

44. S. W. Lane, D. T. Scadden, and D. G. Gilliland, The leukemic stem cell niche: current concepts and therapeutic opportunities, Blood 114, 1150-1157 (2009).

1. An in vitro cultured human bone marrow microenvironment comprising a scaffold coated with human mesenchymal stem cells and a culture medium, wherein the stem cells are viable and proliferate in culture.

2. The microenvironment of claim 1, wherein the medium comprises SDF-1/CXCL12.

3. The microenvironment of claim 1, wherein the scaffold is an elastomeric matrix.

4. The microenvironment of claim 3, wherein the matrix is reticulated and resiliently-compressible.

5. The microenvironment of claim 3, wherein the matrix is porous.

6. The microenvironment of claim 3, wherein the matrix comprises polycarbonate polyurethane.

7. The microenvironment of claim 1, wherein the human mesenchymal stem cells are normal cells.

8. The microenvironment of claim 1, wherein the human mesenchymal stem cells are taken from a patient.

9. The microenvironment of claim 1, wherein the mesenchymal stem cells are negative for hematopoietic (CD34, CD45) markers and endothelial (CD11b, CD14 and CD31) lineage associated markers.

10. The microenvironment of claim 1, wherein the mesenchymal stem cells are positive for CD29, CD44, CD73, CD105, CD106 and CD166.

11. The microenvironment of claim 1, wherein the mesenchymal stem cells are positive for CD90, CD105 and CD146 phenotypic markers.

12. The microenvironment of claim 1, wherein a fraction of the mesenchymal stem cells express CXCR4.

13. The microenvironment of claim 1, wherein a fraction of the mesenchymal stem cells comprises a rapidly replicating subpopulation of mesenchymal stem cells.

14. The microenvironment of claim 1, which further comprises leukemia cells.

15. The microenvironment of claim 14, wherein the leukemia cells are taken from a patient's bone marrow, peripheral blood or leukapheresis harvest.

16. The microenvironment of claim 14, wherein the leukemia cells are human acute myeloid leukemia cells.

17. The microenvironment of claim 16, wherein the human acute myeloid leukemia cells are CD45 positive.

18. The microenvironment of claim 16, wherein the human acute myeloid leukemia cells before introduction into the scaffold are CD34 and CD38 negative.

19. The microenvironment of claim 16, wherein a portion of the human acute myeloid leukemia cells express CXCR4.

20. The microenvironment of claim 16, wherein the acute myeloid leukemia cells are Ki-67 positive or Ki-67 negative.

21. A non-human animal comprising a scaffold coated with human mesenchymal stem cells introduced into the animal ectopically.

22-50. (canceled)

51. A method of making the in vitro human bone marrow microenvironment of claim 1 comprising culturing a scaffold with human mesenchymal stem cells under conditions permitting the stem cells to coat the scaffold.

52-54. (canceled)

55. A method of making the non-human animal model of claim 21 comprising the steps of:
a) culturing a scaffold with human mesenchymal stem cells under conditions permitting the stem cells to coat the scaffold; and
b) introducing the scaffold coated with the human mesenchymal stem cells into the non-human animal ectopically.

56-58. (canceled)

59. A method for evaluating an agent for anti-leukemic properties comprising the steps of:
a) obtaining or preparing the in vitro human bone marrow microenvironment of claim 14;
b) contacting the agent with the microenvironment; and
c) evaluating the anti-leukemic properties of the agent.

60-66. (canceled)

67. A method for evaluating an agent for anti-leukemic properties comprising the steps of:
a) obtaining or preparing the non-human animal of claim 42;
b) introducing the agent into the non-human animal; and
c) evaluating the anti-leukemic properties of the agent.

68-75. (canceled)
